(19) United States
(12) Patent Application Publication
(10) Pub. No.: US 2008/0319527 A1
(43) Pub. Date: Dec. 25, 2008

(54) SHAPED MULTI-DUROMETER FILLER

(76) Inventors: Jeffrey A. Lee, Plymouth, MN (US); Steven J. Ferry, Excelsior, MN (US)

Correspondence Address: SCHWEGMAN, LUNDBERG & WOESSNER, P.A., P.O. BOX 2938, MINNEAPOLIS, MN, 55402, US

(21) Appl. No.: 11/767,277

(22) Filed: Jun. 22, 2007

(51) Int. Cl. A61F 2/06

(52) U.S. Cl. 623/1.15

(57) ABSTRACT

Embodiments of the invention include an aneurysm filler, comprising a main body having a transport shape and three-dimensional shape which is manifest when the main body is deployed in an aneurysm.

### FIELD

The inventive subject matter described herein relates to a shaped multi-durometer aneurysm filler and to a method for repairing an aneurysm. The inventive subject matter also relates to a method for making, a method for using the shaped multi-durometer aneurysm filler.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the products, processes and data as described below and in the tables that form a part of this document: Copyright 2007, Neurovasx, Inc. All Rights Reserved.

### BACKGROUND OF THE INVENTION

An aneurysm is a balloon-like swelling in a wall of a blood vessel. Aneurysms result in weakness of the vessel wall in which it occurs. This weakness predisposes the vessel to tear or rupture with potentially catastrophic consequences for any individual having the aneurysm. Vascular aneurysms are a result of an abnormal dilation of a blood vessel, usually resulting from disease and/or genetic predisposition which can weaken the arterial wall and allow it to expand. Aneurysm sites tend to be areas of mechanical stress concentration so that fluid flow seems to be the most likely initiating cause for the formation of these aneurysms.

Aneurysms in cerebral circulation tend to occur in an anterior communicating artery, posterior communicating artery, and a middle cerebral artery. The majority of these aneurysms arise from either curvature in the vessels or at bifurcations of these vessels. The majority of cerebral aneurysms occur in women. Cerebral aneurysms are most often diagnosed by the rupture and subarachnoid bleeding of the aneurysm.

Cerebral aneurysms are most commonly treated in open surgical procedures where the diseased vessel segment is clipped across the base of the aneurysm. While considered to be an effective surgical technique, particularly considering an alternative which may be a ruptured or re-bleed of a cerebral aneurysm, conventional neurosurgery suffers from a number of disadvantages. The surgical procedure is complex and requires experienced surgeons and well-equipped surgical facilities. Surgical cerebral aneurysm repair has a relatively high mortality and morbidity rate of about 2% to 10%.

Current treatment options for cerebral aneurysm fall into two categories, surgical and interventional. The surgical option has been the long held standard of care for the treatment of aneurysms. Surgical treatment involves a long, delicate operative procedure that has a significant risk and a long period of postoperative rehabilitation and critical care. Successful surgery allows for an endothelial cell to endothelial cell closure of the aneurysm and therefore a cure for the disease. If an aneurysm is present within an artery in the brain and bursts, this creates a subarachnoid hemorrhage, and a possibility that death may occur. Additionally, even with successful surgery, recovery takes several weeks and often requires a lengthy hospital stay.

In order to overcome some of these drawbacks, interventional methods and prostheses have been developed to provide an artificial structural support to the vessel region impacted by the aneurysm. The structural support must have an ability to maintain its integrity under blood pressure conditions and impact pressure within an aneurysmal sac and thus prevent or minimize a chance of rupture. U.S. Pat. No. 5,405,379 to Lane, discloses a self-expanding cylindrical tube which is intended to span an aneurysm and result in isolating the aneurysm from blood flow. While this type of stent-like device may reduce the risk of aneurysm rupture, the device does not promote healing within the aneurysm. Furthermore, the stent may increase a risk of thrombosis and embolism. Additionally, the wall thickness of the stent may undesirably reduce the fluid flow rate in a blood vessel. Stents typically are not used to treat aneurysms in a bend in an artery or in tortuous vessels such as in the brain because stents tend to straighten the vessel.

U.S. Pat. No. 5,354,295 to Guglielmi et al., describes a type of vasoclusion coil. Disadvantages of use of this type of coil are that the coil may compact, may migrate over time, and the coil does not optimize the patient's natural healing processes.

### IN THE FIGURES

FIG. 1 illustrates a perspective view of a scaffold and filler wound around and secured to the scaffold.

FIG. 2 illustrates a side view of a filler that has been imparted with a three-dimensional shape but has not been heat set.

FIG. 3 illustrates a side view of the filler of FIG. 2 that has been impacted with a three-dimensional shape but has been heat set.

FIG. 4 illustrates a multidurometer AF filler embodiment.

FIG. 5 illustrates a side view of an extruded, tapered, multidurometer core.

FIG. 6 is another embodiment of an extruded, tapered, multidurometer core.

### DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the aneurysm filler embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition levels are in reference to the active level of that component or composition, and are exclusive of impurities, for example, residual solvents or by-products, which may be present in commercially available sources.

As used herein, the term “multi-durometer” refers to a polymeric material having different degrees of hardness along its area or volume.

Embodiments of the invention described herein include a multi-durometer, preshaped polymeric aneurysm filler. The pre-shaping produces a three-dimensional filler which, when deployed from a catheter, takes on a predetermined shape. In one embodiment, the polymeric material used to make the filler includes varying durimeter features of one type of polymer or, for other embodiments, includes a variety of durometers of multiple materials reformed in aggregate to form a filler shaft. A benefit of using multiple materials is that a greater number of materials' properties is available for use.

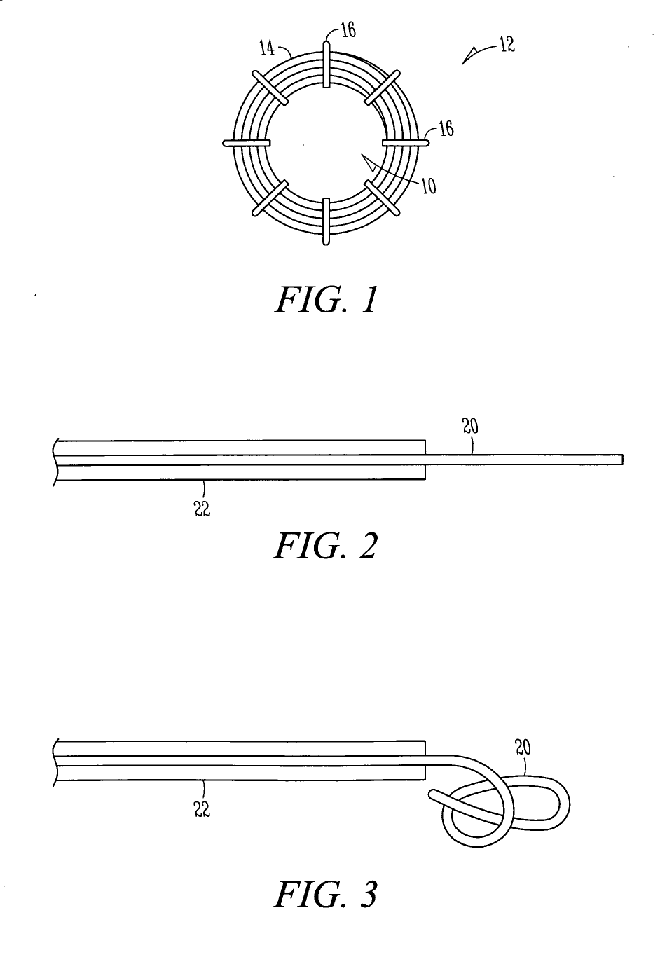

For some embodiments, the polymeric filler is formed into a three-dimensional shape. The method includes a use of a scaffold that includes but is not limited to a sphere, rod, square, triangle, rectangle, or any other geometric shape that can be fabricated. The scaffold is, for some embodiments, made of a metal or suitable polymer. For some embodiments, pins 16, shown in FIG. 1, are inset in a predetermined pattern on the surface of the scaffold to give a desired three-dimensional effect. FIG. 1 illustrates a scaffold 10 and AF filler 12 wound around the scaffold 10 and secured to the scaffold 10. The filler 12 is formed by the scaffold 10 to a sphere 14.

The sphere 14 is placed in or near a heat source in order to raise the temperature of the filler material to its glass transition temperature point, Tg. The glass transition temperature was held for a time effective to produce a heat set. When the heating time expired, the sphere and scaffold were allowed to cool. Once cooled, the filler material was removed from the scaffold and a mandrel was carefully inserted into the filler while being straightened. A reason for artificially straightening is that the filler has taken the heat set.

Once a mandril is in place, the filler can be processed as shown in FIGS. 2 and 3. FIG. 2 shows a filler 20 that has been imparted with a three-dimensional shape but has not been heat set. FIG. 3 shows the filler 20 that has been imparted with a three-dimensional shape and has been heat set.

The aneurysm filler embodiments may be formed by one or more processes such as reflow, thermal welding, adhesive welding, extrusion processing or other mechanisms for attaching two or more materials together.

Another embodiment includes a filler that includes two or more materials of varying durometers forming the length of the filler. The materials may be the same with differing durometers or may be different but compatible materials that posses certain desirable features such as compliance (filling, packing, geometry); lubricity; radiopacity; improved tracking; improved strength and improved healing response.

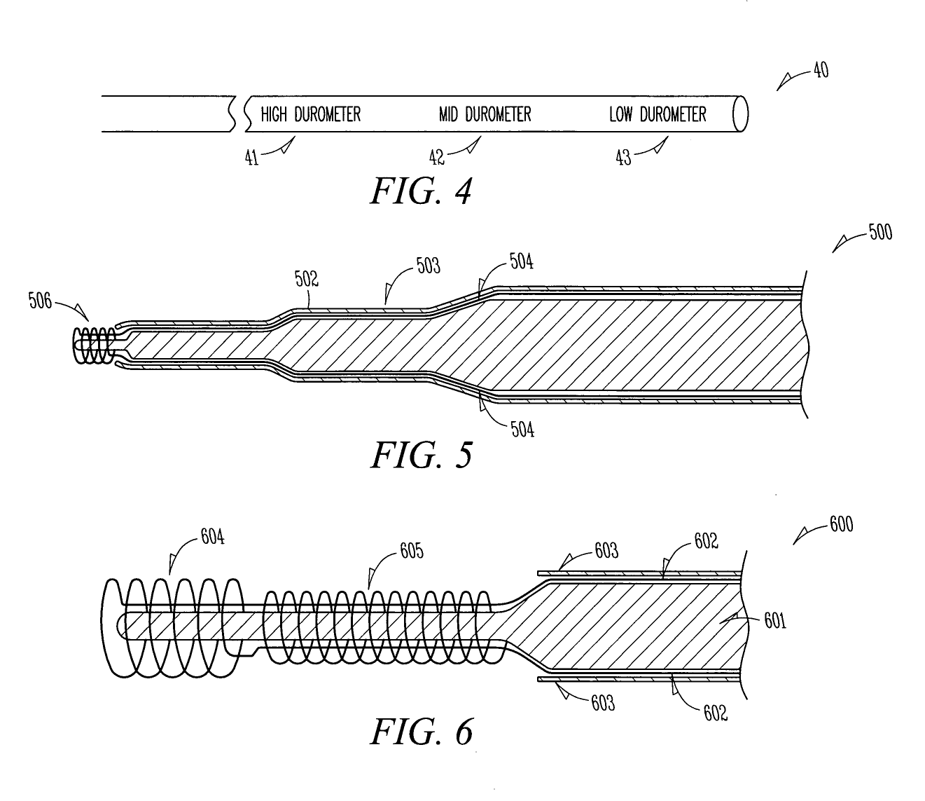

One method of fabrication includes multi-head and multi-durometer extrusion and reflow of material durometers using a heat mandril and appropriate heat shrink materials. One example of this filler embodiment is shown at 40 in FIG. 4. The filler 40 includes a high durometer region 41, a mid durometer region 42 and a low durometer region 43.

Other components of this embodiment include a non fill pusher shaft, a filler midshaft and a distal filler shaft.

An embodiment of an extruded, tapered, multidurometer core made of a material such as PEEK or Teflon is illustrated at 500 in FIG. 5. The embodiment 500 includes an extruded core 502 with embedded leads or laminated leads, shown at 504. The leadwires 504 provide a closed circuit system. The extruded core 500 also includes a heatshrink/reflowed layer 503 to keep the leadwires 504 in place. The extruded multidurometer core 500 also includes a heater coil 506.

One other embodiment of an extruded, tapered, multidurometer core is shown at 600 in FIG. 6. This core embodiment 600 also includes an extruded, tapered, multidurometer core 601 and leadwires providing a closed circuit system 602. The core 600 also includes a heatshrink reflowed layer which keeps the leadwires 602 in place. The embodiment 600 also includes a heater coil 604, but the heater coil 604 is much larger than the heater coil 506. The embodiment 600 also includes an intermediate coil for trackability that extends from the heater coil 605 to the most proximal taper.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

What is claimed is:

1. An aneurysm filler, comprising a main body having a transport shape and three-dimensional shape which is manifest when the main body is deployed in an aneurysm.

2. The aneurysm filler of claim 1, wherein the main body comprises varying durometers of the same material.

3. The aneurysm filler of claim 1, wherein the main body comprises an aggregate of materials of varying durometers.

4. A method for making an aneurysm filler, comprising:
providing a scaffold having a pre-selected three-dimensional shape wherein the scaffold includes pins inset to impart a pre-selected three-dimensional effect; and
positioning filler material about the scaffold and pins.

5. The method of claim 4, further comprising heating the scaffold, pins and filler material to the glass transition temperature of the filler material.

6. The method of claim 5, further comprising cooling the scaffold, pins and filler material.

7. The method of claim 6, further comprising removing the filler material from the scaffolding.

8. The method of claim 6, further comprising straightening the filler material.

9. A scaffold for shaping aneurysm filler material, comprising:
a scaffold main body having a pre-selected shape; and
pins inset in the scaffold main body that impart a three-dimensional effect to the aneurysm filler material.

10. The scaffold main body of claim 9, having a shape selected from the group consisting of spheres, rods, squares, triangles, pyramids, and rectangles.

11. The scaffold main body comprising metal or structural polymer.

12. A filler comprising a high durometer region, a mid durometer region and a low durometer region.

13. The filler of claim 12, further comprising a pusher shaft.
